(19) United States
(12) Patent Application Publication
(10) Pub. No.: US 2007/0191649 A1
(43) Pub. Date: Aug. 16, 2007

(54) Method for producing a propargyl alcohol and an allyl alcohol

(75) Inventors: Katrin Klass, Mannheim (DE); Thilo Hahn, Freimersheim (DE); Jochem Henkelmann, Mannheim (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/588,486

(22) PCT Filed: Feb. 19, 2005
(86) PCT No.: PCT/EP05/01755

§ 371 Date: Aug. 4, 2006

(30) Foreign Application Priority Data
Feb. 26, 2004 (DE) 102004009311.3

(51) Int. Cl. C07C 29/42

(52) U.S. Cl. 568/879

(57) ABSTRACT

Process for preparing a propargyl alcohol of the formula I 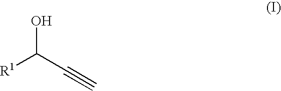
in which R¹ is a C₁₃₀-alkyl, C₃₋₈-cycloalkyl, C₂₋₂₀-alkoxyalkyl, C₆₋₁₄-aryl, C₇₋₂₀-alkoxyarl, C₇₋₂₀-aralkyl, C₇₋₂₀-alkylaryl radical or H, by reacting a corresponding aldehyde of the formula R¹—CHO with acetylene in the presence of ammonia and a catalytic amount of an alkali metal hydroxide, alkaline earth metal hydroxide or alkali metal alkoxide in the range from 0.6 to 10 mol % based on the aldehyde used, and also
processes for preparing an allyl alcohol of the formulae II and III 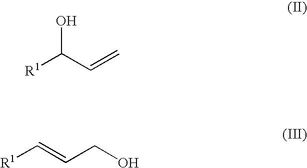 starting from the propargyl alcohol I prepared in accordance with the invention.

The present invention relates to a process for preparing a propargyl alcohol of the formula I 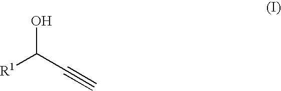
in which R¹ is a C₁₋₃₀-alkyl, C₃₋₈-cycloalkyl, C₂₋₂₀-alkoxyalkyl, C₆₋₁₄-aryl, C₇₋₂₀-alkoxyaryl, C₇₋₂₀-aralkyl, C₇₋₂₀-alkylaryl radical or hydrogen (H), and processes for preparing an allyl alcohol of the formulae II and III 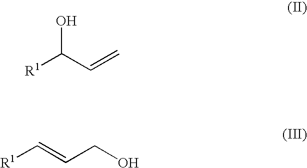
starting from the propargyl alcohol I prepared in accordance with the invention.

The continuous ethynylation of ketones with acetylene in liquid ammonia with catalytic amounts of base (usually KOH or potassium methoxide in a polar, protic solvent; 10 to 40° C.; 20 bar) is described, for example, in DE-B-12 32 573 (SNAM S.p.A.).

The preparation of tertiary propargyl alcohols by reacting ketones, especially methyl alkyl ketones, with acetylene in the presence of NH₃ and a base is also disclosed by EP-A2-1 256 560 (BASF AG).

At partial conversions of only from 50 to 95%, selectivities of >90% are attained.

Base-catalyzed conversions of aldehydes are far more difficult to carry out with high selectivities, since aldehydes have a substantially higher reactivity compared to ketones and lead to undesired by-products, for example aldol condensation products.

Owing to the high reactivity of the aldehydes, the conversion in particular of aldehydes in the presence of a basic catalyst prepared from ammonia and a Brønsted base leads to further by-products such as imines and alpha,beta-unsaturated imines.

For example, when 2-ethylhexanal is used, the imine of the formula 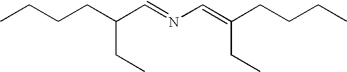
occurs as a by-product.

The ethynylation of 2-ethylhexanal may be carried out continuously in an autoclave at elevated temperature and elevated pressure with stoichiometric amounts of NaOMe in THF (10% by weight solution).

WO 04/018400 (published on Mar. 4, 2004) teaches the preparation of acetylenically unsaturated alcohols by reacting formaldehyde, aldehyde or ketone with acetylene in the presence of ammonia and an alkali metal hydroxide in an amount of less than 1:200 based on the carbonyl compound used.

It is an object of the present invention to find an improved economically viable process for preparing secondary propargyl alcohols. The process should afford the particular propargyl alcohol in high yields and space-time yields at high aldehyde conversions and high selectivities (based on the aldehyde). The high aldehyde conversion (>95%, in particular >98%) makes it unnecessary to recycle unconverted aldehyde into the synthesis, which enables a particularly economically viable method.

[Space-time yields are reported in “amount of product/(volume of catalyst ·time)” (kg/(I_{cat.}·h)) and/or “amount of product/(reactor volume ·time)” (kg/(I_{reactor}·h)].

Accordingly, a process has been found for preparing a propargyl alcohol of the formula I 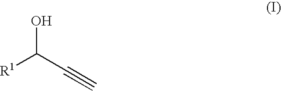
in which R¹ is a C₁₋₃₀-alkyl, C₃₋₈-cycloalkyl, C₂₋₂₀-alkoxyalkyl, C₆₋₁₄-aryl, C₇₋₂₀-alkoxyaryl, C₇₋₂₀-aralkyl, C₇₋₂₀-alkylaryl radical or H, which comprises reacting a corresponding aldehyde of the formula R¹—CHO with acetylene in the presence of ammonia and a catalytic amount of an alkali metal hydroxide, alkaline earth metal hydroxide or alkali metal alkoxide in the range from 0.6 to 10 mol % based on the aldehyde used.

In addition, a process has been found for preparing an allyl alcohol of the formula II 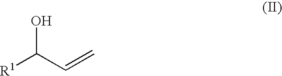
in which R¹ is as defined above, which comprises preparing a propargyl alcohol of the formula I by a process as described above and then reacting with hydrogen in the presence of a hydrogenation catalyst.

Furthermore, a process has been found for preparing an allyl alcohol of the formula III 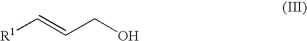
in which R¹ is as defined above, which comprises preparing an allyl alcohol of the formula II by a process as described above and then carrying out a 1,3-allyl rearrangement.

Unexpectedly, it has been found that the more reactive aldehydes R¹—CHO in comparison to the process using methyl ketones described in EP-A2-256 560 (BASF AG) can be ethynylated to the corresponding propargyl alcohols I at higher conversion and higher selectivity and it is thus possible to dispense with costly and inconvenient recyclings, resulting from partial conversion, or at least distinctly reduce the recycle streams.

The process according to the invention for preparing a propargyl alcohol of the formula I can be performed as follows.

The ethynylation can be carried out batchwise or preferably continuously, for example in tubular reactors or else autoclaves.

The reaction is generally carried out at temperatures in the range from 0 to 50° C., in particular from 10 to 40° C.

In general, the reaction is effected at absolute pressures in the range from 1 to 30 bar, in particular from 15 to 25 bar, for example at 20 bar.

The aldehyde R¹—CHO and the acetylene are generally used in a molar ratio in the range of aldehyde:acetylene=from 1:1 to 1:10, preferably aldehyde:acetylene=from 1:2 to 1:4.

The catalytic amount of alkali metal hydroxide, alkaline earth metal hydroxide or alkali metal alkoxide is preferably in the range from 0.8 to 10 mol %, more preferably in the range from 1 to 10 mol % and in particular in the range from 2 to 5 mol %, based on the aldehyde used.

For the catalyst, it is possible to use any alkali metal hydroxide (alkali metal=Li, Na, K, Rb, Cs), alkaline earth metal hydroxide (alkaline earth metal=Be, Mg, Ca, Sr, Ba) or alkali metal alkoxide (alkali metal=Li, Na, K, Rb, Cs). However, preference is given to sodium methoxide, potassium methoxide, sodium hydroxide and in particular potassium hydroxide. The use of potassium methoxide reduces the formation of by-products even further.

When a catalytic amount of an alkali metal alkoxide is used, the alkoxide is preferably a C₁₋₄-alkoxide.

The hydroxide and the alkoxide may be used as a solution or suspension in a solvent such as an alcohol (e.g. C₁₋₄-alcohol such as methanol, ethanol, n-propanol, n-butanol) or an ether (e.g. THF, MTBE).

The alkali metal alkoxide is preferably dissolved in the alcohol which corresponds to the alkoxide by protonation.

The molar ratio of acetylene to ammonia which is present fully or partly in liquid form or in liquid phase under the reaction conditions is generally in the range from 3:7 to 3:16, in particular in the range from 3:7 to 3:12.

In the process according to the invention, the yields based on the aldehyde used, depending on reaction time which is generally in the range from 10 min to 1 h, are very high (from 85 to 97%), especially virtually quantitative (from >97 to 100%).

The degrees of conversion are also good even within quite short time intervals; after about 30 hours, a conversion (an aldehyde conversion) of >95%, in particular from 96 to 99%, can be achieved.

In a particular embodiment, the reactor is charged via metering pumps with a solution of acetylene in ammonia, for example, from a stock vessel and a catalyst solution from another stock vessel. The aldehyde is metered from a third stock vessel in the desired ratios.

In this preferred method, the aldehyde is not initially dissolved in ammonia and the base (e.g. KOH, potassium alkoxide or sodium alkoxide) subsequently added.

Rather, it has been found to be advantageous when all reaction partners are mixed simultaneously. This may be achieved, for example, by dissolving acetylene in ammonia, for example using a static mixer, and subsequently simultaneously metering in all reaction partners (acetylene in ammonia, solution of the hydroxide or alkoxide, aldehyde), for example via a mixing junction.

In this process variant, conversion to propargyl alcohol is accordingly effected by simultaneously metering a stream comprising acetylene and ammonia, a stream comprising the aldehyde and a stream comprising the alkali metal hydroxide, alkaline earth metal hydroxide or alkali metal alkoxide into the reactor.

R¹ may be the following radicals:

H (hydrogen),

C₁₋₃₀-alkyl, especially C₁₋₁₄-alkyl, such as methyl, ethyl, n-propyl, isopropyl, n-butyl, iso-butyl, sec-butyl, tert-butyl, n-pentyl, isopentyl, sec-pentyl, neopentyl, 1,2-dimethylpropyl, n-hexyl, isohexyl, sec-hexyl, cyclopentylmethyl, n-heptyl, isoheptyl, 3-heptyl, cyclohexylmethyl, n-octyl, isooctyl, 2-ethylhexyl, n-decyl, 2-n-propyl-n-heptyl, n-tridecyl, 2-n-butyl-n-nonyl and 3-n-butyl-n-nonyl,

C₃₋₈-cycloalkyl such as cyclopropyl, cyclobutyl, cyclopentyl, cyclohexyl, cycloheptyl and cyclooctyl,

C₂₋₂₀-alkoxyalkyl, more preferably C₂₋₈-alkoxyalkyl, such as methoxymethyl, ethoxymethyl, n-propoxymethyl, isopropoxymethyl, n-butoxymethyl, isobutoxymethyl, sec-butoxymethyl, tert-butoxymethyl, 1-methoxyethyl and 2-methoxyethyl, in particular C₂₋₄-alkoxyalkyl,

C₆₋₁₄-aryl, such as phenyl, 1-naphthyl, 2-naphthyl, 1-anthryl, 2-anthryl and 9-anthryl, preferably phenyl, 1-naphthyl and 2-naphthyl,

C₇₋₂₀-alkoxyaryl, such as o-, m- or p-methoxyphenyl and o-, m- or p-ethoxyphenyl,

C₇₋₂₀-aralkyl, preferably C₇₋₁₂-phenylalkyl, such as benzyl, p-methoxybenzyl, 3,4-di-methoxybenzyl, 1-phenethyl, 2-phenethyl, 1-phenylpropyl, 2-phenylpropyl, 3-phenylpropyl, 1-phenylbutyl, 2-phenylbutyl, 3-phenylbutyl and 4-phenylbutyl, and

C₇₋₂₀-alkylaryl, preferably C₇₋₁₂-alkylphenyl, such as 2-methylphenyl, 3-methylphenyl, 4-methylphenyl, 2,4-dimethylphenyl, 2,5-dimethylphenyl, 2,6-dimethylphenyl, 3,4-dimethylphenyl, 3,5-dimethylphenyl, 2,3,4-trimethylphenyl, 2,3,5-trimethylphenyl, 2,3,6-trimethylphenyl, 2,4,6-trimethylphenyl, 2-ethylphenyl, 3-ethylphenyl, 4-ethylphenyl, 2-n-propylphenyl, 3-n-propylphenyl and 4-n-propylphenyl.

The aldehydes of the formula R¹—CHO used in the process according to the invention are in particular those where R¹=C₄₋₁₀-alkyl or phenyl, such as 2-ethylhexanal, n-hexanal and benzaldehyde.

Preference is also given to using aldehydes which have a carbon branch at the alpha-carbon atom.

The alcohols prepared with preference by the ethynylation process according to the invention are in particular 4-ethyloct-1-yn-3-ol, oct-1-yn-3-ol and 3-phenyl-1-propyn-3-ol.

Employable processes and catalysts for the selective hydrogenation of alkynes to alkenes, especially propargyl alcohols to allyl alcohols, are known to those skilled in the art. For example, reference is made to the prior art disclosed in EP-A1-827 944 and EP-A2-1 256 560 (both BASF AG).

To increase the selectivity, carbon monoxide (CO) may be added to the hydrogen. The hydrogenation catalysts used comprise in catalytically active metal of transition group VIII of the Periodic Table of the Elements, preferably Pd, and optionally further elements such as elements of main group III, IV, V, VI and/or of transition group I, II, III, VI, VII of the Periodic Table of the Elements for doping.

The catalysts are preferably thin-layer catalysts which are prepared, for example, by vapor deposition or sputtering (see, for example, EP-A-564 830 and EP-A-412 415) or preferably by impregnation (see, for example, EP-A-827 944 and EP-A1-965 384). However, the catalysts may also be used in the form of other shaped bodies, for example extrudates or tablets.

Very suitable as active components and support materials are those mentioned in EP-A-827 944. The outer shape of the catalysts is likewise described in EP-A-827 944 and the references cited therein.

In a particular embodiment, the selective, preferably continuous, hydrogenation of the alkynes is carried out in liquid phase over thin-layer catalysts using hydrogen or a gas mixture which, in addition to hydrogen, may comprise small amounts of CO. Based on EP-A2-1 256 560, the hydrogenation is preferably carried out in a system composed of two reactors (main reactor and postreactor), if appropriate with recyclings, at elevated pressure and elevated temperature.

The thin-layer catalysts preferably comprise palladium as the active metal and, if appropriate, one or more promoters, for which Ag and Bi are preferred. The thin-layer catalysts are preferably prepared by impregnating a metal fabric or knit with a solution which comprises active metal and, if appropriate, promoters. The thin-layer catalysts are preferably used in the form of monoliths, which may be prepared, for example, in accordance with EP-A-827 944 from the support material before or after the impregnation.

Employable processes and catalysts for the selective 1,3-allyl rearrangement of secondary allyl alcohols to primary allyl alcohols are also known to those skilled in the art. For example, reference is made to the prior art disclosed in WO-A1-02/24617 (BASF AG) and the sources cited there.

The alcohols prepared with preference by the ethynylation process according to the invention in conjunction with partial hydrogenation and, if appropriate, 1,3-allyl rearrangement are in particular 4-ethyloct-1-en-3-ol, oct-1-en-3-ol, 3-phenylprop-1-en-3-ol and cinnamyl alcohol (3-phenyl-2-propen-1-ol).

The purification of the alcohols prepared by the process according to the invention is preferably distillative, for example also in dividing wall columns.

The product alcohols of the process according to the invention find use, for example, in fragrances or as lubricants in oil wells.

### EXAMPLES

1. Ethynylation and partial hydrogenation of 2-ethylhexanal

2-Ethylhexanal (2-EH) (purity: 98.9 GC area %) was reacted with acetylene and catalytic amounts of potassium methoxide in methanol (32% by weight) in liquid ammonia to give the corresponding acetylene alcohol ethyloctynol. The active catalyst is probably a potassium acetylide complex stabilized by ammonia. All reaction partners were simultaneously mixed in a mixing junction. In a second stage, the acetylene alcohol formed, ethyloctynol, was partially hydrogenated over a thin-layer catalyst using hydrogen to give the corresponding allyl alcohol, ethyloctenol. The analysis for this example, unless stated otherwise, was carried out using gas chromatography.

In detail: a) Ethynylation in the presence of NH₃/KOMe (continuous plant):

The reactor used was a 1073 ml stainless steel reactor having plug flow characteristics (reaction tube having an internal diameter of 6 mm). 330 g/h of 2-ethylhexanal, 179 I(STP)/h of acetylene (I(STP)=liters at STP=volume converted to standard conditions), 688 g/h of NH₃ and 8.2 g/h of potassium methoxide solution in methanol (32% by weight) were pumped continuously into the reactor. All three streams were metered under mass flow control into the reactor. Acetylene was dissolved in ammonia using a mixer before it was metered into the reactor. Stoichiometries of the feeds:

Metering: 2-EH/NH₃/C₂H₂/KOMe=1 /15.9/3.1/0.015 (calculated in [mol/mol of aldehyde]),

Residence time: 30.5 min, temperature profile: reactor inlet 38° C., reactor outlet 34° C. The reaction discharge was under pressure control (20 bar+/−0.05 bar). The degassing was effected in three stages:

1. Flash vessel at 90° C., 1013 mbar

2. Thin-film evaporator at 50° C., 1013 mbar

3. Degasser at 40° C., 150 mbar

The neutralization and hydrolysis were effected with 307 g/h of water and 2.5 I (STP)/h of CO₂ gas in a mixer at 75° C. After phase separation in a coalescence filter (50 μm) at 70° C., the organic phase was dried in a further thin-film evaporator which was operated at 85° C. and 70 mbar. 400 g/h of organic effluent (>97 GC area % of ethyloctynol, up to 1.3 GC area % of the corresponding acetylenediol) were continuously passed on into the hydrogenation stage. The aqueous phase removed contained, in addition to potassium hydrogencarbonate, traces of ammonium hydrogencarbonate.

b) Partial hydrogenation:

The experiment was carried out in a continuous apparatus having two tubular reactors. The first reactor was operated in liquid phase mode with recycling at a liquid superficial velocity of 200 m³/m²/h and a hydrogen superficial velocity of 200 m³/m²/h at a total pressure of 7 bar. The cycle gas was injected via a driving jet nozzle. Sufficient CO was added to the hydrogen in the first reactor that the CO concentration in the cycle gas was from 300 to 500 ppm. The temperature in the first reactor was 94° C. The feed rate to the first reactor of crude ethyloctynol was 300-400 g/h. In the first reactor, a Pd thin-layer catalyst with Ag doping was used and had a metal content of 280 mg of Pd/m² and 70 mg of Ag/m² on Kanthal fabric (materials number 1.4767). The second reactor was operated in liquid phase mode in straight pass at 5.5 bar and 76° C. The feed rate of effluent from the first reactor was controlled via the level of a gas-liquid separator. In the second reactor, a Pd thin-layer catalyst having Bi doping was used. The effluent of the second reactor was passed on continuously to distillative workup. In the continuous hydrogenation, a selectivity of at least 96.4% based on 4-ethyloct-1-en-3-ol was achieved over a prolonged period at a conversion of at least 99.7%. A maximum of 1.1% of the saturated alcohol, 4-ethyloctan-3-ol (subsequent product of the hydrogenation), was found in the effluent.

The thin-layer catalysts described in this example were obtained by impregnating metal fabric, as described, for example, in EP-A2-1 256 560 (BASF AG).

Balancing of the ethynylation of 2-ethylhexanal:

The ethynylation was used to conduct a total of three mass balances. The following table summarizes the results:

| Balance time | C (ethylhexanal) | S (ethyloctynol) | Y (ethyloctynol) |
|---|---|---|---|
| [h] | [%] | [%] | [%] |
| 48 | 98.4 | 90.9 | 89.5 |
| 120 | 99.5 | 91.6 | 91.2 |
| 120 | 99.4 | 90.9 | 90.4 |

(C = conversion, S = selectivity, Y = yield)

The balance results show that the ethynylation of 2-ethylhexanal can be carried out with very good yields (91.2%) and selectivities (91.6%). In comparison to the ethynylation of ketones, for example tetrahydrogeranylacetone (THGAC) and hexahydrofarnesylacetone (HEX), the aldehyde 2-ethylhexanal is surprisingly virtually 100% converted with high selectivity.

The formation of the imine 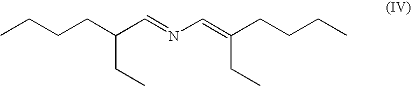
was only observed in the trace region (<0.02 GC area %).

High boiler analysis:

The high boiler determination by reduced pressure Kugelrohr distillation of the ethylhexanal reactant and of effluents from the ethynylation provided no indication of increased high boiler formation: reactant: 0.1% by weight, ethynylation effluents:≦0.2% by weight residue). Nor were any aldol condensation products identified in the GC and GC-MS analysis.

1. A continuous process for preparing a propargyl alcohol of the formula I 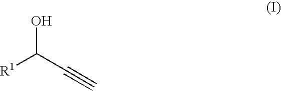
in which R¹is a C₁₋₃₀-alkyl radical branched on the α-carbon atom, which comprises reacting a corresponding aldehyde of the formula R¹—CHO with acetylene in the presence of ammonia and a catalytic amount of an alkali metal hydroxide, alkaline earth metal hydroxide or alkali metal alkoxide in the range from 0.6 to 10 mol % based on the aldehyde used.

2. The process according to claim 1, wherein the reaction is carried out at temperatures in the range from 0 to 50° C.

3. The process according to claim 1, wherein the reaction is carried out at absolute pressures in the range from 1 to 30 bar.

4. The process according to claim 1, wherein the aldehyde and the acetylene are used in a molar ratio in the range of aldehyde:acetylene of from 1:1 to 1:10.

5. The process according to claim 1, wherein the catalytic amount of alkali metal hydroxide, alkaline earth metal hydroxide or alkali metal alkoxide is in the range from 1 to 10 mol % based on the aldehyde used.

6. The process according to claim 1, wherein R¹ is a C₄₋₁₀-alkyl radical branched on the α-carbon atom.

7. The process according to claim 1, wherein R¹ is 3-heptyl.

8. The process according to claim 1, wherein conversion to propargyl alcohol is effected by simultaneously metering a stream comprising acetylene and ammonia, a stream comprising the aldehyde and a stream comprising the alkali metal hydroxide, alkaline earth metal hydroxide or alkali metal alkoxide into a reactor.

9. The process according to claim 1, wherein the alkoxide is a C₁₋₄-alkoxide.

10. The process according to claim 1, wherein the alkali metal is sodium or potassium.

11. The process according to claim 1, wherein the alkaline earth metal is magnesium or calcium.

12. The process according to claim 1, wherein the alkali metal alkoxide or metal hydroxide is dissolved or suspended in an alcohol.

13. The process according to claim 12, wherein the alkali metal alkoxide is dissolved or suspended in the alcohol that corresponds to the alkoxide by protonation.

14-18. (canceled)
